(19) United States
(12) Patent Application Publication
(10) Pub. No.: US 2010/0120087 A1
(43) Pub. Date: May 13, 2010

(54) Enzymatic or Organic Catalytic Chemical Reactions

(75) Inventors: Osman Kibar, San Diego, CA (US); Mirianas Chachisvilis, San Diego, CA (US); Eugene Tu, San Diego, CA (US)

Correspondence Address: FISH & RICHARDSON PC, P.O. BOX 1022, MINNEAPOLIS, MN, 55440-1022, US

(73) Assignee: Dynamic Connections, LLC

(21) Appl. No.: 12/693,190

(22) Filed: Jan. 25, 2010

Related U.S. Application Data
(63) Continuation-in-part of application No. 12/543,190, filed on Aug. 18, 2009.
(60) Provisional application No. 61/171,645, filed on Apr. 22, 2009.
(60) Provisional application No. 61/172,838, filed on Apr. 27, 2009.
(60) Provisional application No. 61/172,959, filed on Apr. 27, 2009.
(60) Provisional application No. 61/179,233, filed on May 18, 2009.
(60) Provisional application No. 61/090,028, filed on Aug. 19, 2008.

(51) Int. Cl. C12P 1/00; C25B 5/00; B01J 19/00; C12M 1/00; B01J 19/08

(52) U.S. Cl. 435/41; 204/157.15; 422/129; 435/289.1; 422/186.04

(57) ABSTRACT

In an aspect, a perturbation is applied to a system comprising an enzymatic chemical reaction and/or an organic catalytic chemical reaction with the perturbation being non-directional (average of the force applied by the perturbation being zero) with respect to a variable of the system. A directional effect is caused with respect to the said enzymatic or organic catalytic chemical reaction as a result of the perturbation and an asymmetry of the perturbed system. The present invention also embodies an apparatus comprising a site for an enzymatic or organic catalytic chemical reaction and a device controlled to perturb a system that includes the enzymatic or organic catalytic chemical reaction, the average of the force applied by the perturbation being zero, a directional effect being caused with respect to the chemical reaction as a result of the perturbation and an asymmetry of the perturbed system.

This application is entitled to the benefit of the priority of U.S. provisional application Ser. 61/171,645, filed Apr. 22, 2009, U.S. provisional application Ser. 61/172,838, filed Apr. 27, 2009, U.S. provisional application Ser. 61/172,959, filed Apr. 27, 2009, and U.S. provisional application Ser. 61/179,233, filed May 18, 2009, and of international application serial PCT/US 2009/054177, filed Aug. 18, 2009, and is a continuation in part of U.S. application Ser. No. 12/543,190, filed Aug. 18, 2009, which has the benefit of priority of each of the above cited provisional applications and also of U.S. provisional application Ser. 61/090,028, filed Aug. 19, 2008. All of the above applications are incorporated here in their entireties by reference.

### BACKGROUND

This description relates to enzymatic and organic catalytic chemical reactions.

In a Brownian motor or pump, for example, a force that is non-directional in a spatial dimension, i.e. the average of the force applied over said spatial dimension is zero, generates a directional motion of particles in the system along that spatial dimension.

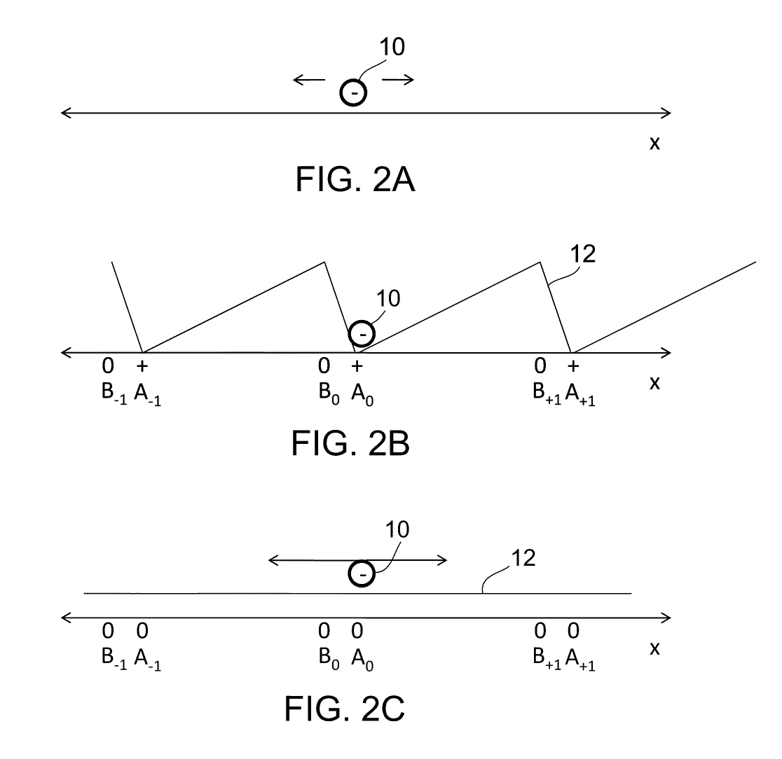

In FIGS. 2A, 2B, and 2C, for example, assume that a negatively charged particle 10 (e.g., a molecule in a liquid) is in thermal equilibrium with its environment. In a one-dimensional system, the molecule can move only along the x-axis, i.e., to the right or to the left in FIG. 2A.

An external electric field (constant in time) is applied to the system to create an energy profile 12 for the molecule as shown in FIG. 2B. In the case of a negatively charged molecule, a positive voltage is applied at each point along the x-dimension that corresponds to a minimum in energy (e.g., A₀) and a zero voltage is applied to each point that corresponds to a maximum in energy (e.g., B₀). When this field is being applied, the molecule will move towards a point of minimum energy, A₀. In the example shown, the A points and the B points are located periodically along the axis and the distance between each A point and the next adjacent B point to its left is less than the distance from that A point to the next adjacent B point to its right.

Once the molecule has had enough time to move to the point of minimum energy, the field is turned off. Then, the energy profile 12 for the molecule is flat as shown in FIG. 2C. With no energy barriers to either side, the molecule will experience Brownian motion and diffuse to either side with equal probability, with the molecule's diffusion away from point A₀ constituting a perturbation of the system.

The field is kept off until the probability of the molecule diffusing at least a distance (B₀−A₀) is significant, but not as much as diffusing at least a distance (B₊₁−A₀). Then the electric field is turned back on to restore the energy profile of FIG. 2B. At this point, if the molecule has diffused to a point to the left of B₀, then it will move (slide down the energy profile) towards A₋₁ under the influence of the electric field. On the other hand, if the molecule had diffused to the right beyond B₊₁, the restored field would cause it to move (slide down the energy profile) towards A₊₁.

However, because the distance (B₀−A₀) is shorter than the distance (B₊₁−A₀), at the instant the field is turned on, the probability of the particle being to the left of B₀ will be greater than of being to the right of B₊₁. In other words the probability is higher that the molecule will have taken a step to the left than to the right.

When this cycle (of turning the field on and off) is repeated many times, the molecule will, on average, have taken more steps to the left than to the right, and therefore have experienced directional motion to the left, even though the applied force (induced by the electric field) is not directional when averaged over space.

Such directional motion can even overcome an opposing force (or a load) on the molecule's motion (i.e., a force tending to push the molecule to the right). The applied non-directional force of the electric field and the non-directional diffusion would be doing directional work, pushing the molecule to the left despite the opposing force.

Brownian ratchet mechanisms have been applied in Brownian motors and Brownian pumps. In the former case, work is done on a particle to move it in a directional manner, e.g., against an opposing force. In the latter case, work is done to pump particles (e.g., ions) against a concentration and/or a voltage gradient). A review on Brownian motor or pumps can be found in various articles (e.g., Astumian and Derenyi, “Fluctuation driven transport and models of molecular motors and pumps”, European Biophysics Journal, vol 27, pp 474-489, 1998, and the references mentioned in that article).

### SUMMARY

In general, in an aspect, a perturbation is applied to a system comprising an enzymatic and/or an organic catalytic chemical reaction, the perturbation being non-directional (average of the force applied by the perturbation being zero) with respect to a variable of the system, and. A directional effect is caused with respect to the said enzymatic or organic catalytic chemical reaction as a result of the perturbation and an asymmetry of the perturbed system.

Implementations may include one or more of the following features. The perturbation is internal to the system. The perturbation is applied externally to the system. The perturbation is varying in space. The perturbation is varying in time. The perturbation is varying in both space and time. The perturbation is deterministic. The perturbation has a profile that is sinusoidal. The perturbation has a profile that is a square wave. The perturbation has a profile that is piecewise linear. The perturbation has a profile that is arbitrary and is not sinusoidal, square wave or piecewise linear. The perturbation has a profile that is a weighted sum of these different profiles. The perturbation is stochastic. The perturbation distribution function has a statistical profile that is Gaussian. The perturbation distribution function has a statistical profile that is Poisson. The perturbation distribution function has a statistical profile that is Lorentzian. The perturbation distribution function has a statistical profile that is different than Gaussian, Poisson or Lorentzian. The perturbation distribution function has a statistical profile with a continuous probability distribution. The perturbation distribution function has a statistical profile with a discrete probability distribution. The perturbation has a profile that is symmetric (f(x+n)=f(−x+n)). The perturbation has a profile that is antisymmetric (−f(x+n)=f(−x+n)). The perturbation has a profile that is asymmetric. The perturbation and/or the system response to the perturbation is centered at zero. The perturbation and/or the system response to the perturbation is biased to a non-zero value. An influence biases the perturbation. An influence biases the system response. An influence biases both the perturbation and the system response. An environmental parameter such as temperature, pressure, or volume biases the perturbation. An environmental parameter biases the system response. An environmental parameter biases both the perturbation and the system response. The perturbation is applied as a digital signal. The perturbation is applied as an analog signal. The perturbation is applied as a profile that may vary with only one parameter. The perturbation is applied as a profile that may vary with two parameters. The perturbation is applied as a profile that may vary with more than two parameters. The perturbation may be in the form of at least one of an electric field, a magnetic field, a substrate concentration, a product concentration, pH, pressure, temperature, an acoustic field, or an electromagnetic field, to interact with a part of a chemical system and modulate its energy levels.

The asymmetry is intrinsic to the system. The asymmetry is extrinsic to the system. The asymmetry is a combination of intrinsic and extrinsic to the system. The asymmetry is spatial, temporal, spatio-temporal, or energetic. The asymmetry is permanent or induced or a combination of the two.

The enzymatic/organic catalytic chemical reaction comprises a surface reaction, a bulk reaction, or a membrane reaction, or combinations of them. The enzymatic/organic catalytic chemical reaction comprises a spontaneous reaction (exothermic) or a non-spontaneous reaction (endothermic), or a combination of the two. The enzymatic/organic catalytic chemical reaction comprises a single chemical pathway or multiple chemical pathways. The enzymatic/organic catalytic chemical reaction is manipulated by controlling a direction of the reaction. The enzymatic/organic catalytic chemical reaction is manipulated by changing a substrate concentration or a product concentration or a ratio of concentrations. The enzymatic/organic catalytic chemical reaction is manipulated by doing work on the system that the system would otherwise not do including against or along other influences and/or gradients. The enzymatic/organic catalytic chemical reaction is manipulated by catalyzing the reaction. The enzymatic/organic catalytic chemical reaction is manipulated by applying one or more of specific enhancement and/or suppression of reactions and/or pathways. The chemical reaction includes a single step reaction or a single pathway. The chemical reaction includes a multiple step reaction or multiple pathways. The multiple pathways are sequential. The multiple pathways are parallel. The perturbation is enhanced by running the chemical reaction on a surface or close to a surface.

The method is optimized for the magnitude of the work done. The optimization for work done includes optimizing a frequency of oscillation and/or a field magnitude of the perturbation to maximize a change in effective barrier height. The method is optimized for energy efficiency. The energy efficiency is optimized by applying a two-or-more-parameter loop profile for the perturbation to reduce the irreversibility of the transitions. There is a particular phase relationship between the parameters of the two-or-more-parameter loop. The method is optimized for operation in a desired regime. The operation in a desired regime is optimized by applying a second perturbation to bias/shift the system response. The method is optimized for a particular load amount.

Application of the perturbation is enhanced by influence mediators. The influence mediators are genetically targeted. The mediators include mutated enzymes. The influence mediators are artificially attached. The mediators are attached by attaching beads or chemical groups to enzymes. The influence mediators are naturally occurring modifications. The influence mediators are post-translational modifications of proteins. The influence mediators can be used without a Q-asys method.

The directional effect comprises altering a final substrate concentration, a final product concentration, or a ratio of the two, relative to their equilibrium concentrations. The directional effect comprises increasing, decreasing, or reversing the spontaneity of the reaction (i.e. whether it is a spontaneous reaction or not under a specific set of environmental parameters.). The directional effect comprises changing a probability of a specific pathway and/or product, relative to another alternative pathway or product, to change a yield of the specific pathway and/or product. The directional effect is applied to multiple chemical steps, or multiple chemical pathways.

The directional effect comprises accelerating or decelerating an enzymatic/organic catalytic chemical reaction, a step of a reaction, a chemical pathway and/or a catalyst. The accelerating or decelerating includes turning on or turning off.

The catalyzing comprises manipulating an energy barrier. The catalyzing comprises manipulating a transmission coefficient. The catalyzing comprises modulating an affinity. The catalyzing comprises manipulating a concentration of a substrate, a product, and/or an intermediate state.

A result or an outcome of the method can be used in chemical manufacturing, chemical processing, an industrial application, an energy application, a biological application, a field of chemistry, a field of biology, or a field of biochemistry.

In general, in an aspect, an apparatus comprises a site for an enzymatic/organic catalytic chemical reaction, and a device controlled to perturb a system that includes the enzymatic/organic catalytic chemical reaction, the average of the force applied by the perturbation over time being zero, a directional effect being caused with respect to the chemical reaction as a result of the perturbation and an asymmetry of the perturbed system.

Implementations may include one or more of the following features. The device comprises a controlled voltage, current, temperature, pressure, pH or concentration perturbations. The site includes a surface. The enzymatic/organic catalytic chemical reaction includes an enzyme catalyzed reaction. The enzyme is covalently attached to a surface.

These and other aspects and features, and combinations of them can be expressed as methods, systems, apparatus, program products, compositions, mixtures, products, and in other ways.

Other advantages, features, and aspects will be apparent to those skilled in this art from the following description and claims.

### DESCRIPTION

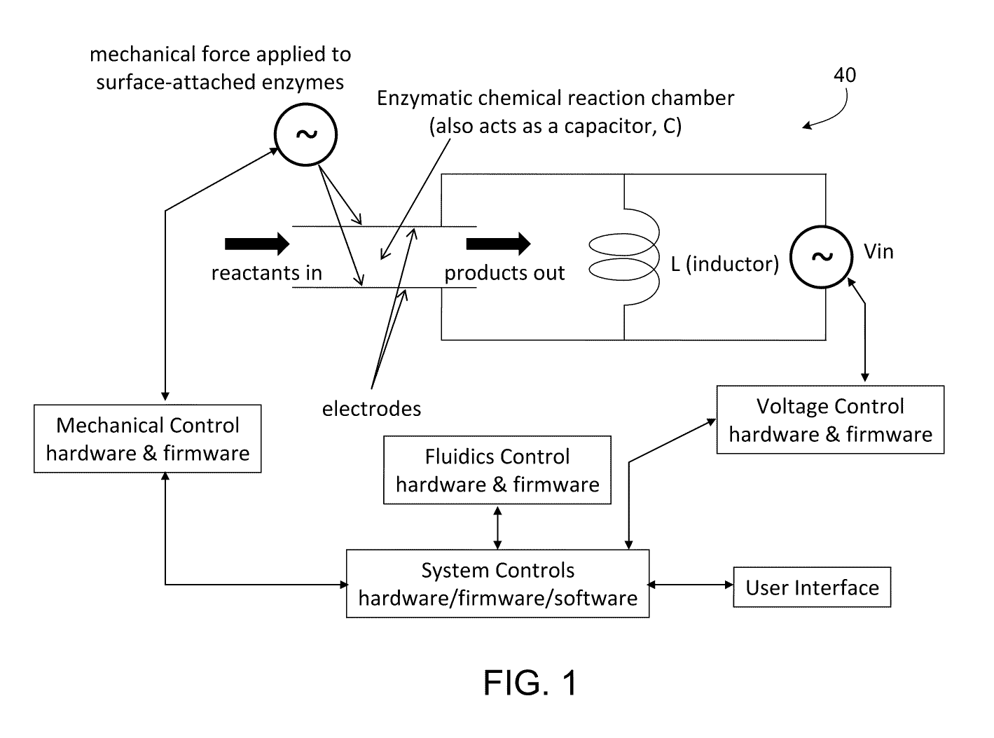

FIG. 1 shows an example apparatus setup.

FIGS. 2A, 2B, and 2C show energy profiles along an axis.

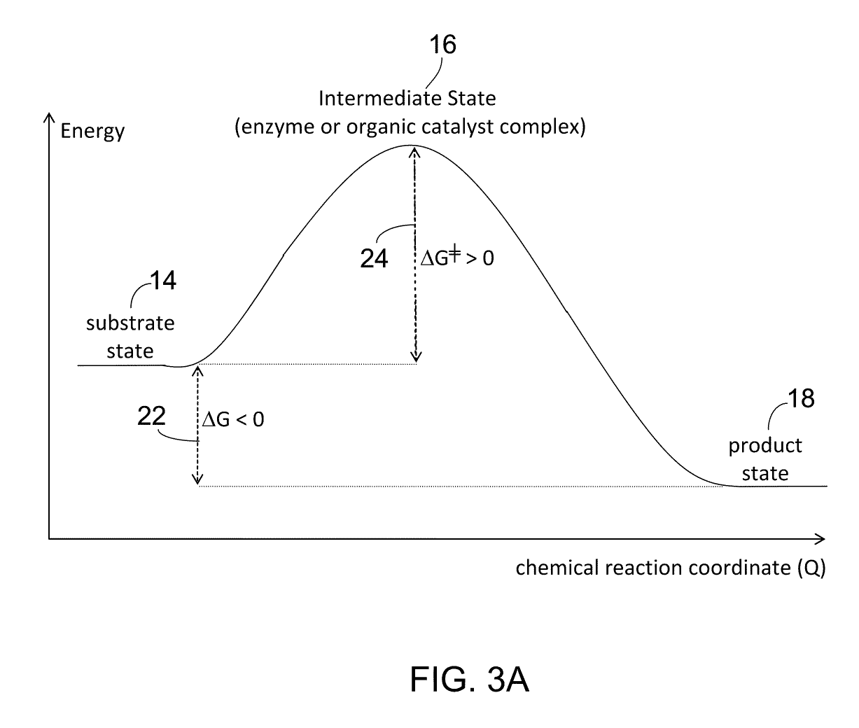

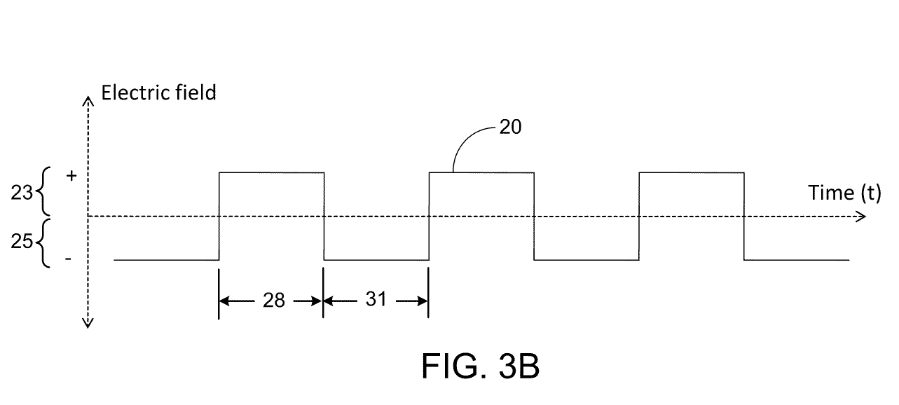

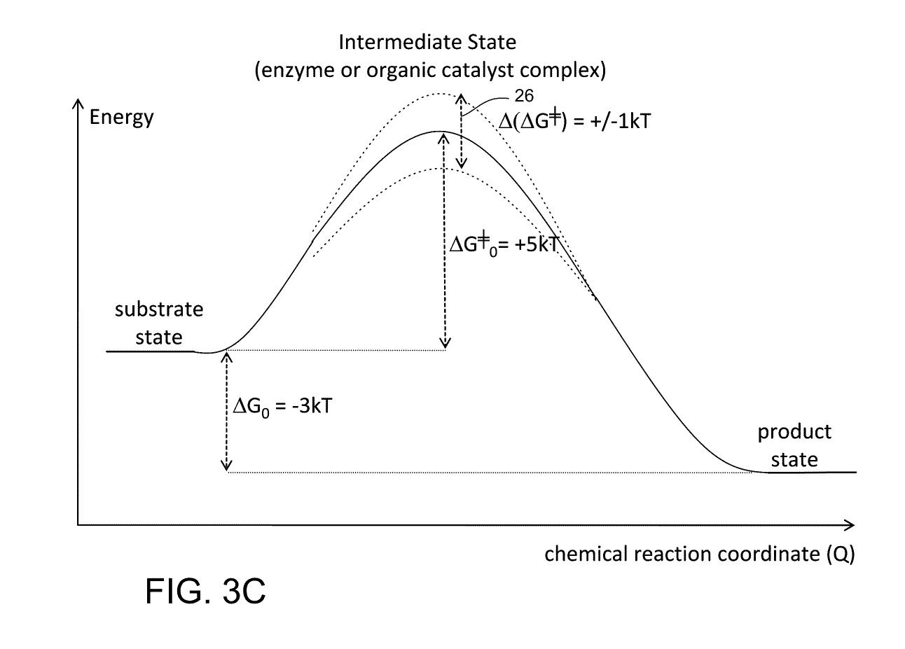

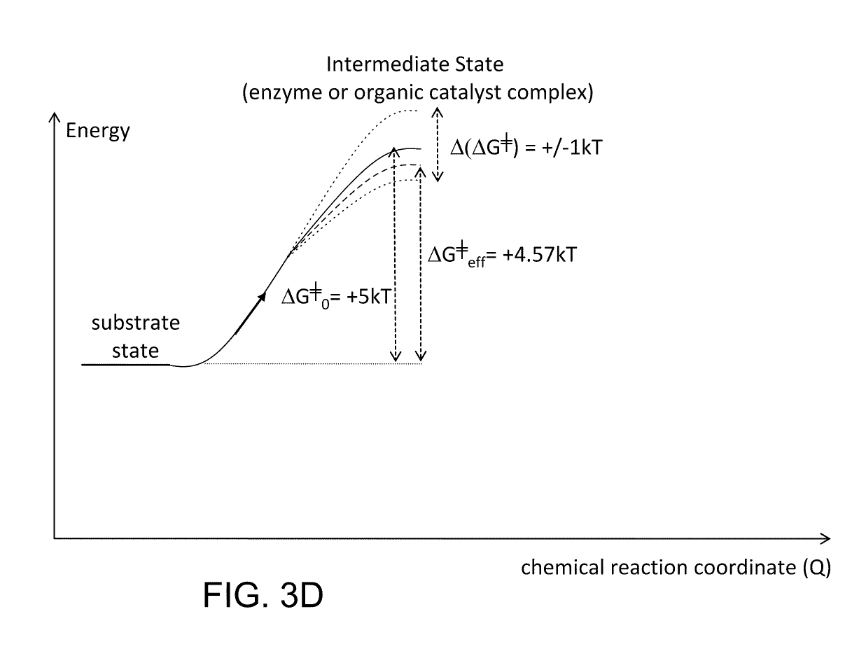

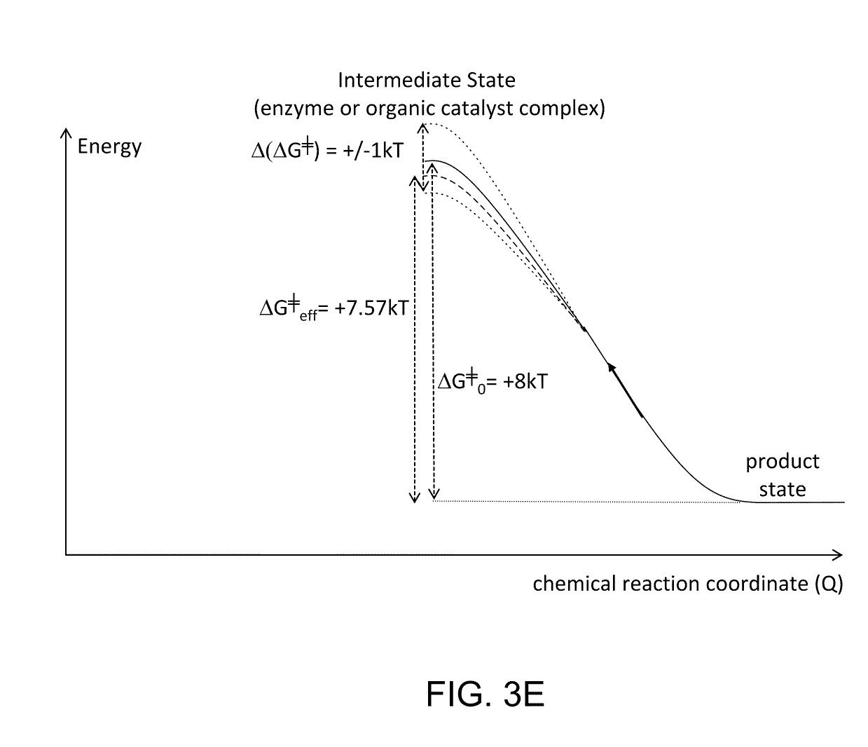

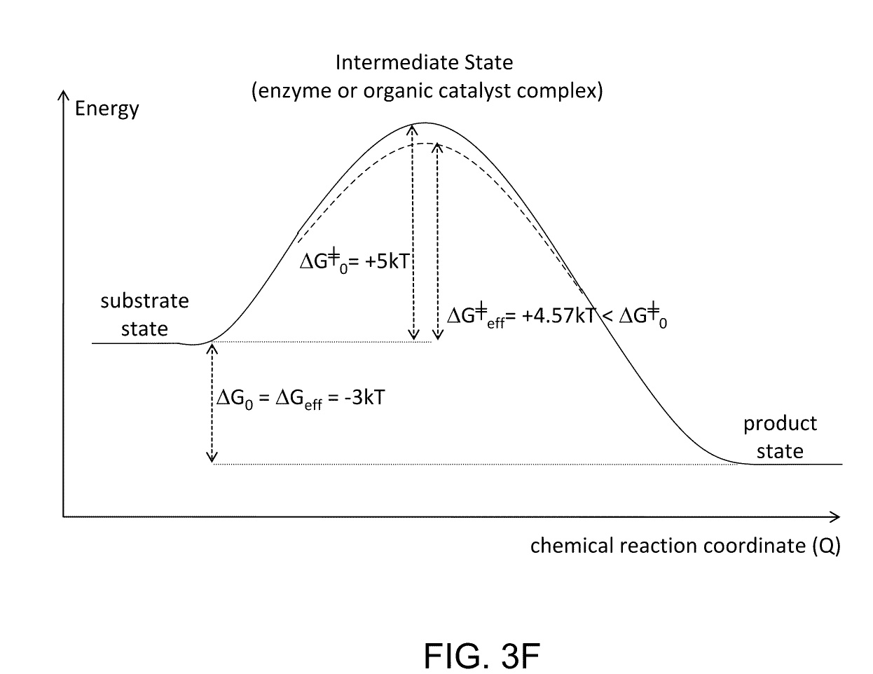

FIGS. 3A, 3C, 3D, 3E, 3F, 4A, 4B, 4C, 8, 9A and 9C are enzymatic or organic catalytic chemical reaction state diagrams.

FIG. 3B is a diagram of an oscillating applied field over time.

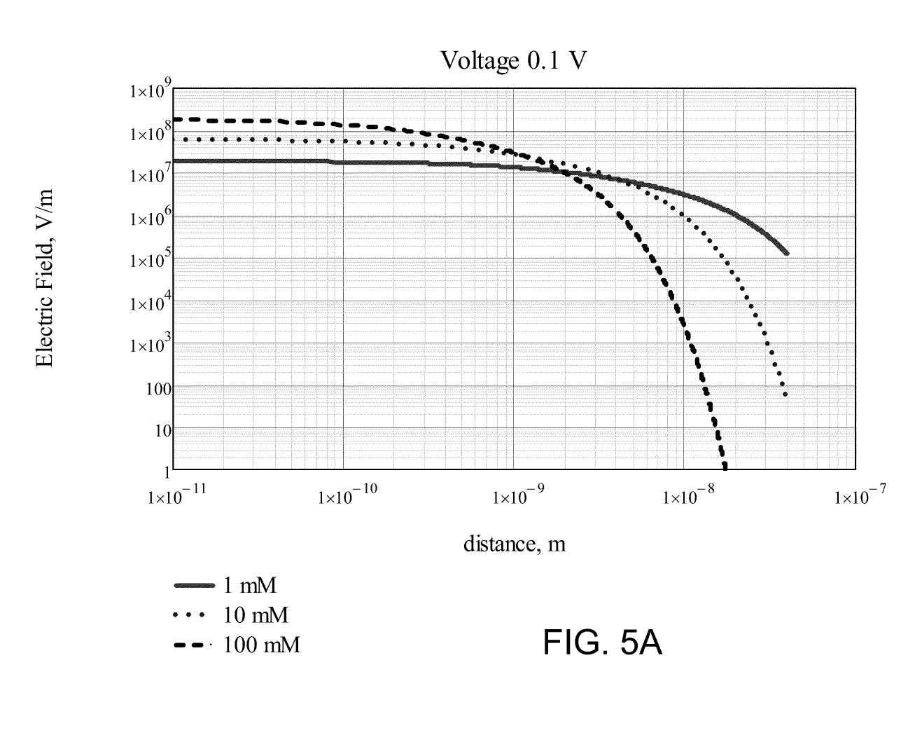

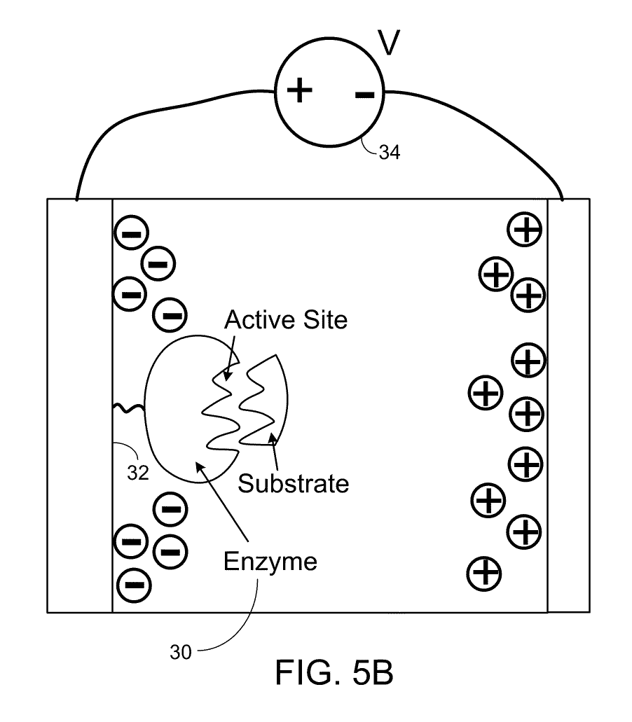

FIGS. 5A and 5B show an example setup, with an oscillating electric field applied to a surface-attached, enzyme-catalyzed chemical reaction.

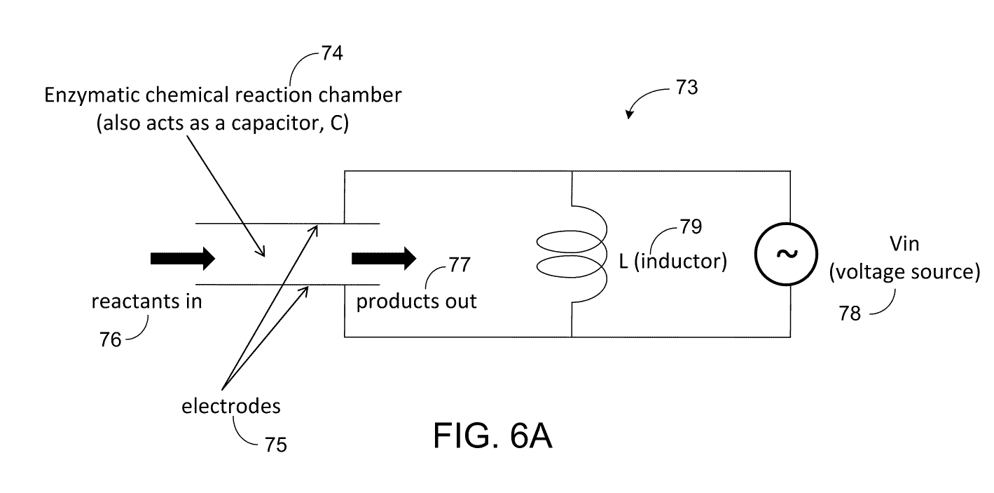

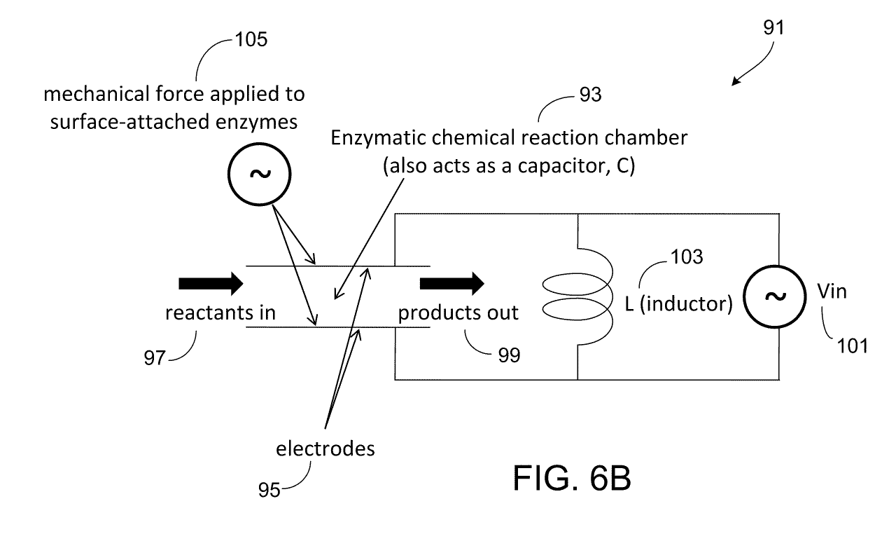

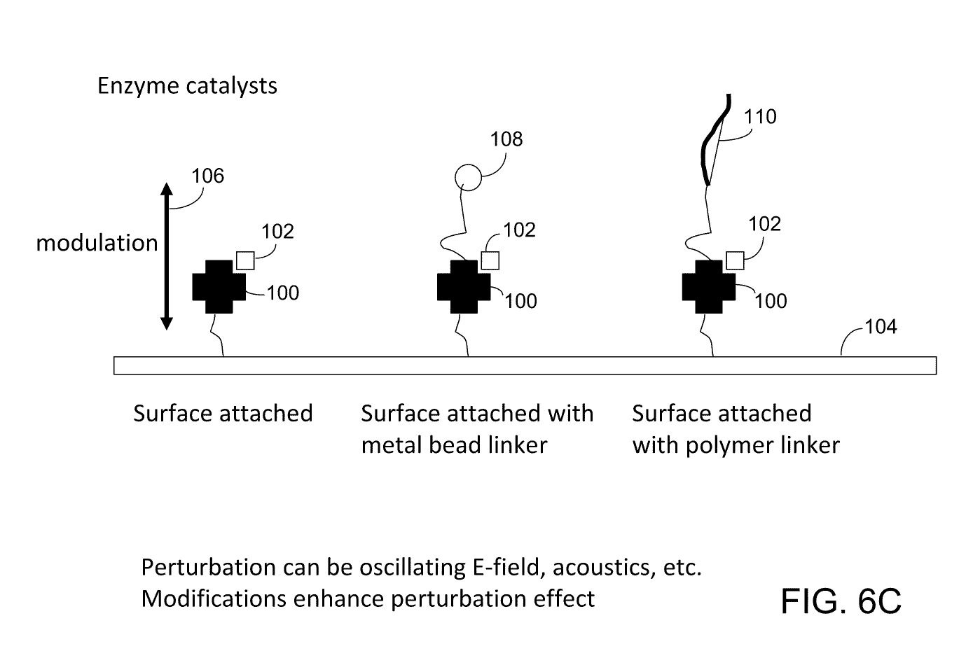

FIGS. 6A and 6B are drawings of apparatus.

FIG. 6C is a schematic diagram of enzymes with polymers.

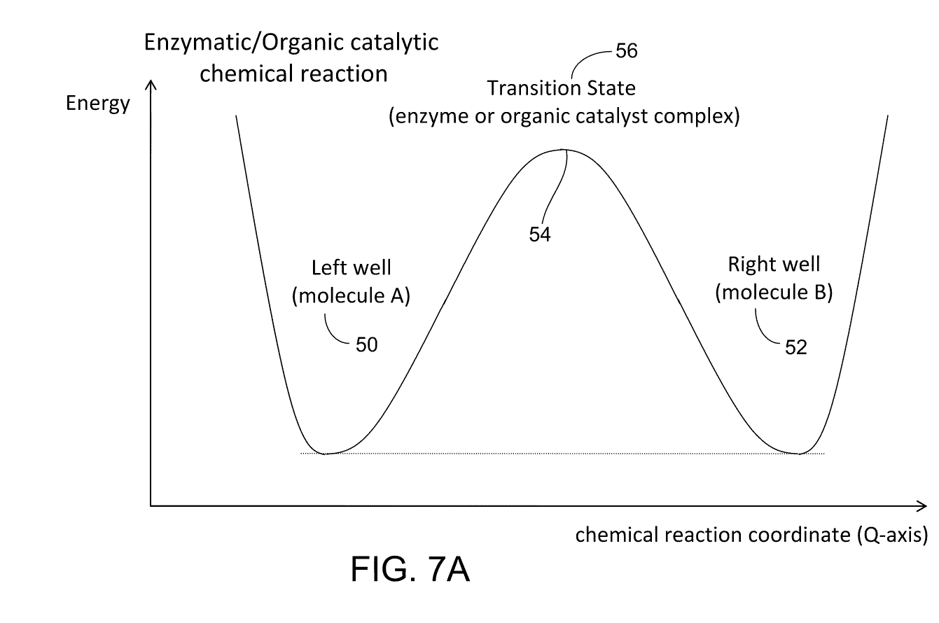

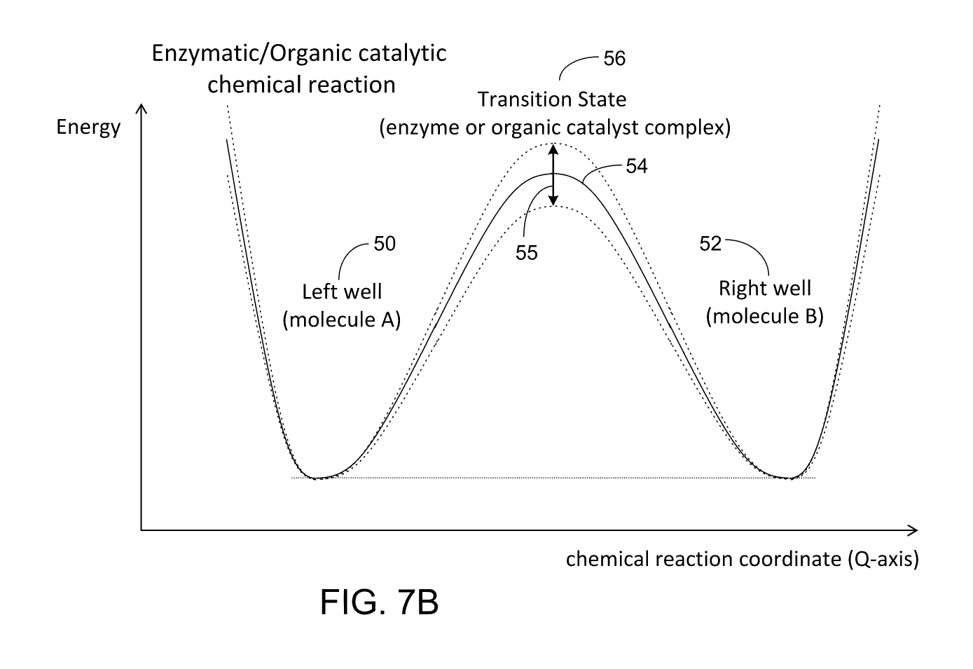

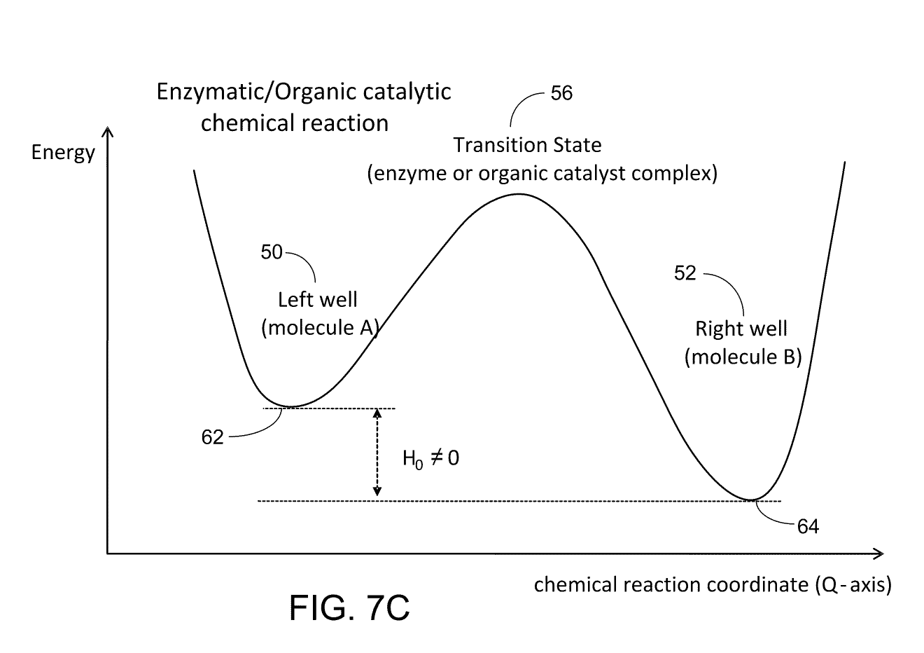

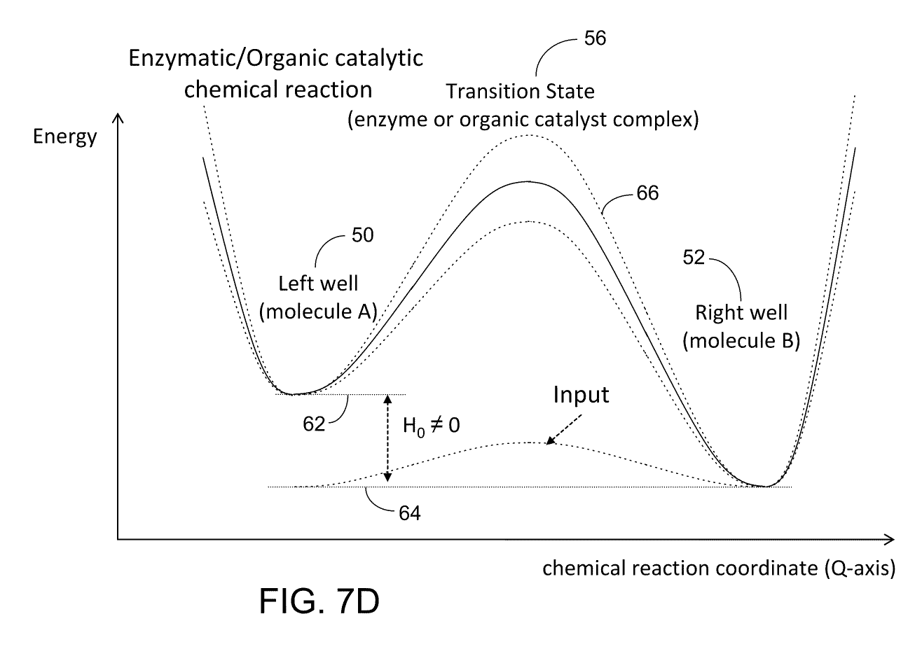

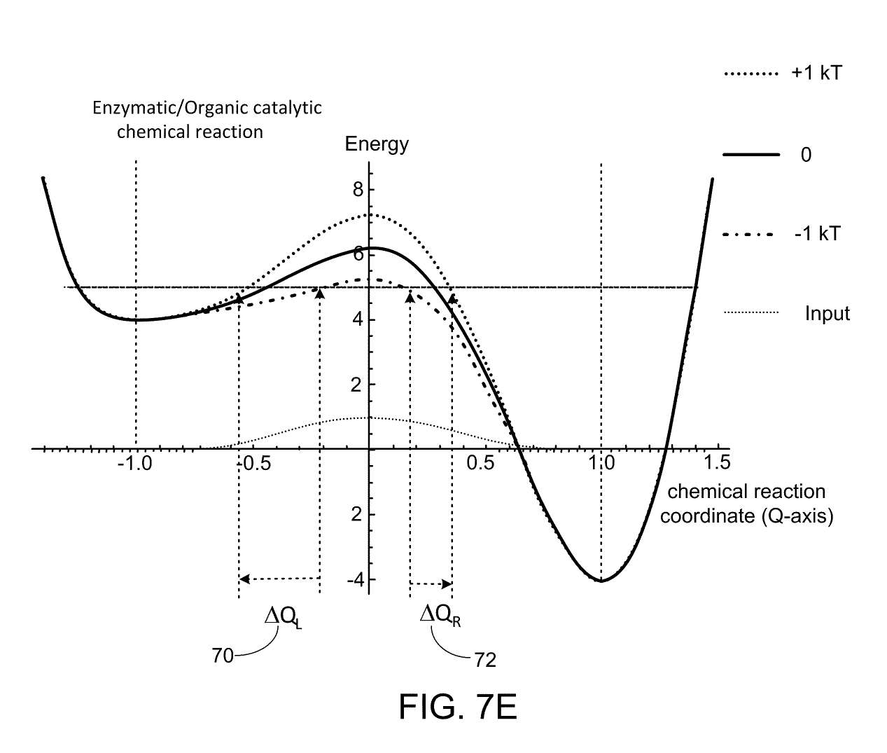

FIGS. 7A, 7B, 7C, 7D and 7E are graphs of energy versus chemical reaction coordinate (Q-space).

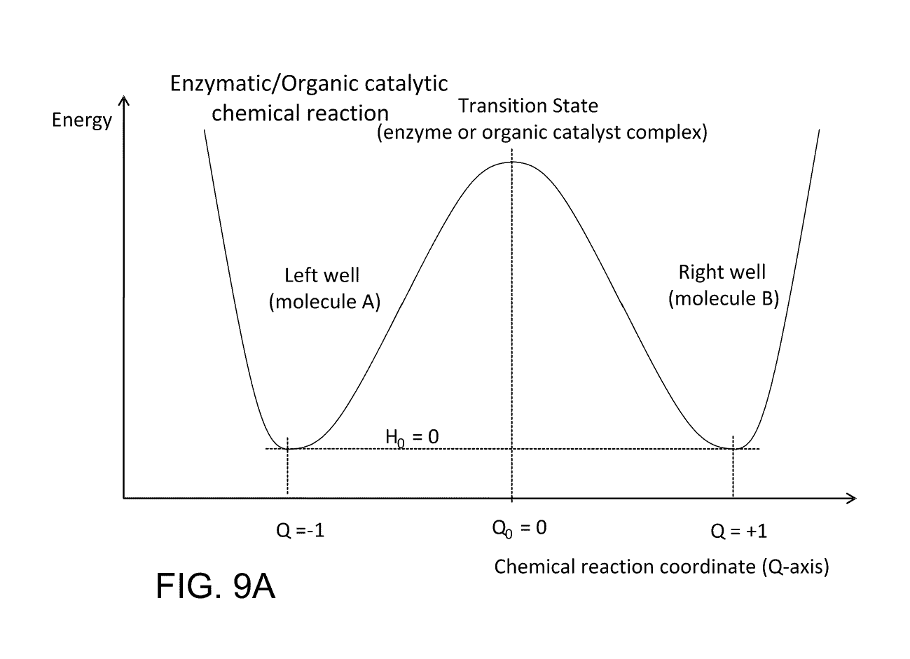

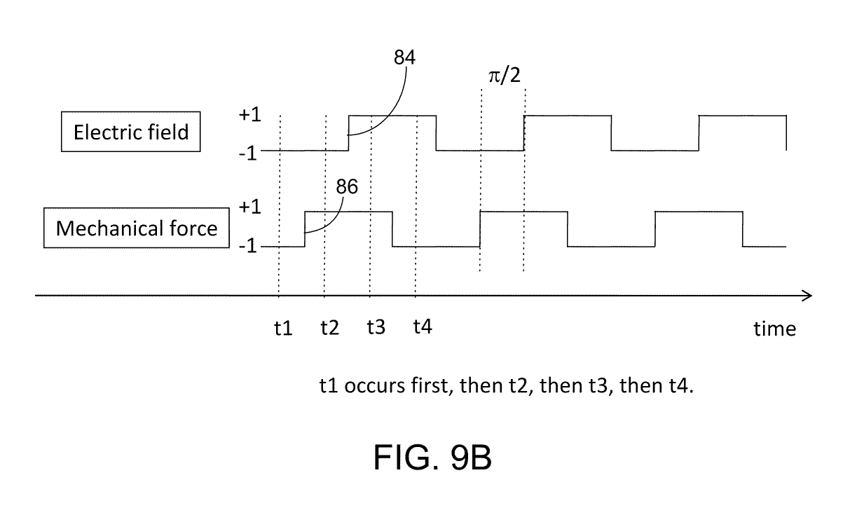

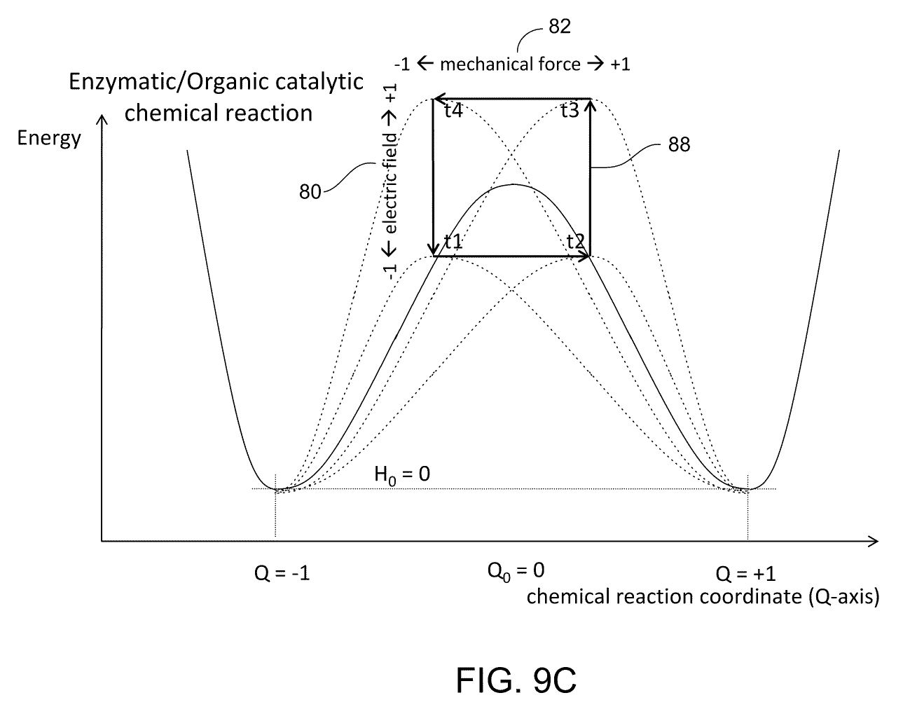

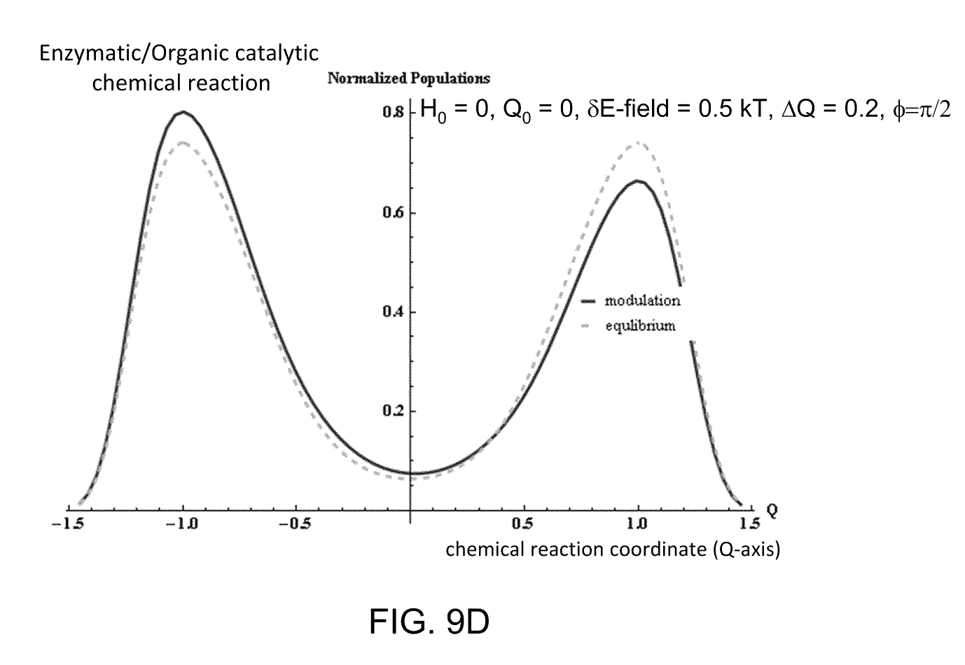

FIG. 9B is a diagram of a two parameter signal waveform over time.

FIG. 9D is a plot of normalized population distribution of molecules versus chemical reaction coordinate (Q-space).

In a Brownian motor or pump, a non-directional force (which has no preferred direction when averaged over a spatial dimension) generates directional motion along that dimension based on two key concepts: perturbation and asymmetry.

Although the force of the electric field is non-directional when averaged over a spatial dimension, when the field is on, the force is directional at individual segments (along the x-axis) and in time at each location along the x-axis (when considered relative to the situation when the field is turned off). In other words, non-directionality does not mean the force is uniform or constant at each point in space or in time. One effect of turning the field (and therefore the force) on and off is to alternately perturb the system from its steady-state in physical space (e.g., move molecules along the x-axis in the figures). In this example, when the field is on, the position of the molecule is stabilized at the minimum energy point. And when the field is off, the system is perturbed by thermal fluctuations, such that the molecule diffuses away from its steady-state position. We sometimes use the terms particle and molecule to refer to a molecule or any other kind of particle or object.

In this application, within the term symmetry, we very broadly include, for example, any invariance of values (e.g., a lack of any perceptible change) under a transformation over a range of interest. Also, within the term antisymmetry, we very broadly include, for example, a symmetry in which the values under the transformation are of opposite sign or sense. And, by asymmetry, we mean very broadly, for example, an absence or a violation of a symmetry or of an antisymmetry or of both.

The second concept, asymmetry, refers to the different probabilities of two events occurring in the system. When the field is off, the probability of the molecule diffusing past the energy barrier on the left is higher than of diffusing past the barrier on the right. Therefore, when averaged over long periods of time (and many periods of the field being turned on and off), a particle experiences a net velocity in one direction (in this example, to the left).

The Brownian motor or pump operates with respect to two parameters of the system: position (e.g., along the x-axis) and momentum (e.g., represented by velocity along a momentum axis, which is not shown). A force (thermal fluctuation), that is non-directional along the position axis (i.e., has zero magnitude when averaged over space, in this case, a single axis, but has non-zero magnitude when viewed in local segments of space), perturbs the system along the x-axis. And by virtue of the asymmetry of the system (the asymmetry of the spatial energy profile due to the applied electric field), the non-directional force generates a directional response of the system along the momentum axis (i.e., a net velocity).

The same key concepts of perturbation and asymmetry to convert non-directional input forces into a desired directional effect (which we call the ASYS mechanism) can be used in a system based on other parameters and variables.

For example, an asymmetric system can be perturbed in time within the context of an enzymatic or organic catalytic chemical reaction system with respect to the parameters of energy (the energy levels involved in the enzymatic chemical reaction or the organic catalytic chemical reaction) and/or chemical reaction coordinate (relative locations of reactant, product and/or intermediate states of a chemical reaction along the chemical reaction coordinate, Q-axis).

Within the term enzymatic chemical reaction, we broadly also include organic catalytic chemical reactions. Furthermore, within the term enzyme, we broadly include organic catalysts.

In this use of a perturbed asymmetric system (which we call a Q-asys), a non-directional force applies a temporal perturbation to the system (e.g., pushes a certain state of the system out of its steady-state energy level and/or Q-axis location). As before, by a non-directional force we mean one that, when averaged over time or over Q-axis, has a zero magnitude, and thus, no preferred direction as a direct influence on the system, but when viewed in particular segments of time, is non-constant or non-uniform and therefore is able to perturb the system. This force, in turn, causes the reaction to proceed in a preferred direction (e.g., by lowering certain energy levels while increasing others, or by altering the equilibrium populations of selective molecules), by virtue of an asymmetry in the perturbed system (e.g., different characteristic transition times for the forward and backward paths of a reaction, or different density of state distributions for the reactant and product states).

Although the applied force (which manipulates the energy levels or the reaction coordinate of the enzymatic/organic catalytic chemical reaction and implicitly therefore the preferred direction of reaction in Q-space) is on average non-directional, the application of the force in an ASYS mode (based on the asymmetry) enables the perturbative force to do useful work on the system, in this case, to drive, alter and/or bias some aspect of the enzymatic/organic catalytic chemical reaction, such as its reaction rate or its final product concentrations.

As illustrated in the example of FIG. 3A, the enzymatic/organic catalytic chemical reaction has a substrate state 14 (the energy level of the substrate molecule(s) of the chemical reaction), a product state 18 (the energy level of the product molecule(s) of the chemical reaction), and an intermediate state 16 that lies between the substrate and product states along the chemical reaction coordinate and has a relatively higher energy level that represents an energy barrier. The enzymatic/organic catalytic chemical reaction has a negative Gibbs energy 22 (ΔG<0) (the energy level of the product state is lower than the energy level of the substrate state), that is, the reaction is exothermic and will proceed on its own without any energy input. The positive energy barrier, Δ [symbol] , 24 between the substrate and product states, limits the rate of the reaction. Assume that an enzyme is involved in this reaction, such that the intermediate state (corresponding to the peak of the energy barrier) comprises the enzyme-substrate complex, and that the barrier height takes into account the effect of the enzyme on the rate of the chemical reaction.

An external force is applied that interacts only with the intermediate state and not with the substrate or the product states. In other words, the external force only affects the peak of the energy barrier, but not the free Gibbs energy of the reaction (i.e., the barrier height Δ [symbol] may change, but the free Gibbs energy of the reaction ΔG remains the same). In this example, as shown in FIG. 3B, the applied force is a square wave electric field 20 that has positive (+) and negative (−) values 23, 25 of equal magnitude, and a 50% duty cycle, i.e., equal time periods 28, 31 for + and − states. Assume that the response of the energy level of the intermediate state to this external force is linear. As a frame of reference, as shown in FIG. 3C, assume the barrier height is 5 kT above the substrate energy level, that ΔG equals −3 kT, and that the applied electric field has a magnitude that causes the intermediate state energy level to change, 26, by +/−1 kT. The applied force is non-directional in this case, because it changes the intermediate state energy level by equal amounts (+/−1 kT) and for equal times (50% duty cycle), so its time average is zero, i.e. <Δ(Δ [symbol] )>=0.

The effect of the oscillation of the applied force (which perturbs the system) on the forward and reverse reactions can be analyzed separately with respect to the two directions of reaction. (By forward reaction we mean the one that moves from the substrate state to the product state, and vice versa for the reverse reaction.)

As shown in FIG. 3D, the net effect of the applied force on the forward reaction can be calculated using the Jarzynski equation [Jarzynski, “Nonequilibrium Equality for Free Energy Differences”, Physical Review Letters, vol 78, no 14, pp 2690-2693, 1997], which relates the average of the work done per transition (from the substrate state to the intermediate state) to the free energy of the system, even for systems that are not in equilibrium: <exp(−W/kT)>=exp(−ΔG/kT), Equation 1.

The work done for each transition of the forward reaction is the difference between the energy levels of the substrate state and the intermediate state (as modulated by the electric field). The energy level of the intermediate state changes by +/−1 kT, so the average force applied to the barrier is zero (i.e. <Δ(Δ [symbol] )>=0, where < > means averaging the argument inside the brackets). However, the work done for each trajectory (i.e., a transition from the substrate state to the intermediate state) is weighted by an exponential term (per equation 1). The effect of the electric field on the work done for a transition is different for a positive electric field than for a negative electric field because of the asymmetry in equation 1 (i.e., the exponential weighing). The average work done for all possible transitions including those for both positive electric fields and negative electric fields (and thus, the effective barrier height, Δ [symbol] _{eff}) will be smaller in the presence of the oscillating electric field than without the field: <exp(−W/kT)>=exp(−Δ [symbol] _{eff}/kT). For a barrier height of Δ [symbol] ₀=5 kT and a modulation of Δ(Δ [symbol] )=+/−1 kT, the barrier height oscillates between 4 kT and 6 kT, so <exp(−W/kT)>=0.5(exp(−4)+exp(−6))=exp(−Δ [symbol] _{eff}/kT), so Δ [symbol] _{eff}˜4.57 kT. This means the oscillating external field has lowered the effective barrier height by about 0.43 kT.

As shown in FIG. 3E, the net effect of the applied force on the reverse reaction is determined by assuming the starting point of the reverse reaction to be the product side and considering individual transitions that go from the product state to the intermediate state (i.e., the peak of the energy barrier). Using the same analysis, this time with a barrier height of Δ [symbol] ₀=8 kT and the same modulation of Δ(Δ [symbol] )=+/−1 kT, the barrier height oscillates between 7 kT and 9 kT, so <exp(−W/kT)>=0.5(exp(−7)+exp(−9))=exp(−Δ [symbol] _{eff}/kT), and Δ [symbol] _{eff}˜7.57 kT, which is again ˜0.43 kT lower than the barrier height of the system with no field (Δ [symbol] ₀=8 kT).

Therefore, as shown in FIG. 3F, the oscillating field reduces the effective barrier height, by the same amount for both the forward and the reverse reactions. This static effect is independent of the frequency of the electric field oscillation or the direction of the transition. The static effect does not change the free Gibbs energy of the reaction, ΔG, and therefore does not affect the final equilibrium of the concentrations of the substrates and products, [S]_{final} and [P]_{final}. Reducing the barrier height changes the reaction rate, i.e., catalyzes the reaction, but not the final equilibrium.

The difference in energy between Δ [symbol] _{eff} and Δ [symbol] ₀ for each transition from a substrate state to the intermediate state (i.e., ˜0.43 kT in our example) is provided to the system by the applied electric field. As long as the system response (in this example, the work done for each transition) to the applied force is a non-antisymmetric function (and equation 1 is such a function of the exponential weighing factor), then there will be a non-zero effect on the system by an antisymmetric applied force (i.e. Δ [symbol] _{eff}≠Δ [symbol] ₀), in this case the non-zero effect being the change in the effective energy barrier height of the reaction.

In addition to the static effect, the oscillating electric field produces a kinetic effect that is dependent on the frequency of the electric field oscillation. For a reaction with a positive energy barrier, the mean first passage time (MFPT) is the characteristic timescale that it takes to transition from a lower energy substrate state to a higher energy intermediate state. MFPT is (exponentially) proportional to the barrier height; that is, τ˜exp(Δ [symbol] /kT).

In the example enzymatic or organic catalytic chemical reaction, MFPT for the forward and the reverse reactions will be different, because each one sees a different barrier height: τ_{F}˜exp(Δ [symbol] _{F}/kT)˜exp(5) and τ_{R}˜exp(Δ [symbol] _{R}/kT)˜exp(8), respectively, with the reverse reaction having a longer MFPT than the forward reaction by a factor of ˜exp(3).

Each cycle of the oscillating electric field applied to the intermediate state perturbs the system by changing the intermediate state energy level up and down. Then any time a system is perturbed, it will seek to reestablish its non-perturbed state (if the perturbation were to be turned off), which can be considered an intrinsic restoring force of the system that acts to return the system back to its non-perturbed state.

The relationship between the frequency of oscillation (γ) of the driving force (induced by the electric field) and the characteristic timescale of the transition (τ) implies a phase difference (i.e., a coherence) between the driving force and the restoring force. This phase difference may, depending on the magnitude of the difference between the oscillation frequency and the transition timescale, cause an interference effect, such that the effective barrier height will shift based on the relative magnitude of the oscillation frequency to that of the characteristic timescale [Bier, Derenyi, Kostur and Astumian, “Intrawell relaxation of overdamped Brownian particles”, Physica Review E, vol 59, no 6, pp 6422-6432, 1999].

If γ>>(1/τ), that is, if the oscillation is too fast for the transition to respond to each of the successive cycles before the next cycle occurs, then the barrier height will effectively remain unchanged.

If, on the other hand, γ<<(1/τ), that is, if the oscillation is too slow compared to the characteristic timescale, then the system will effectively experience a higher barrier half the time (for applied field of one polarity) and a lower barrier the other half of the time (for applied field of the opposite polarity). Because the barrier height affects the transition time exponentially, the average (i.e., the effective) barrier height will have increased.

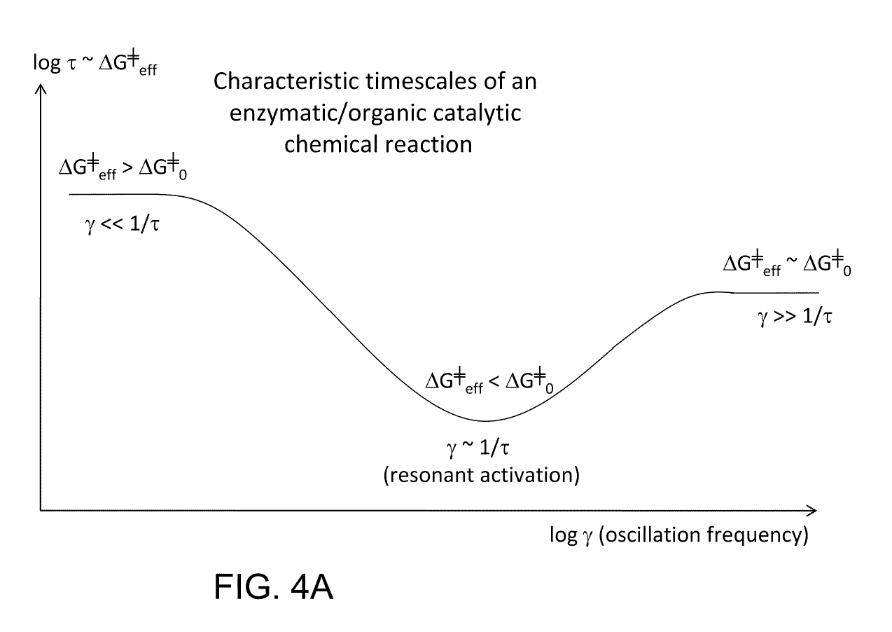

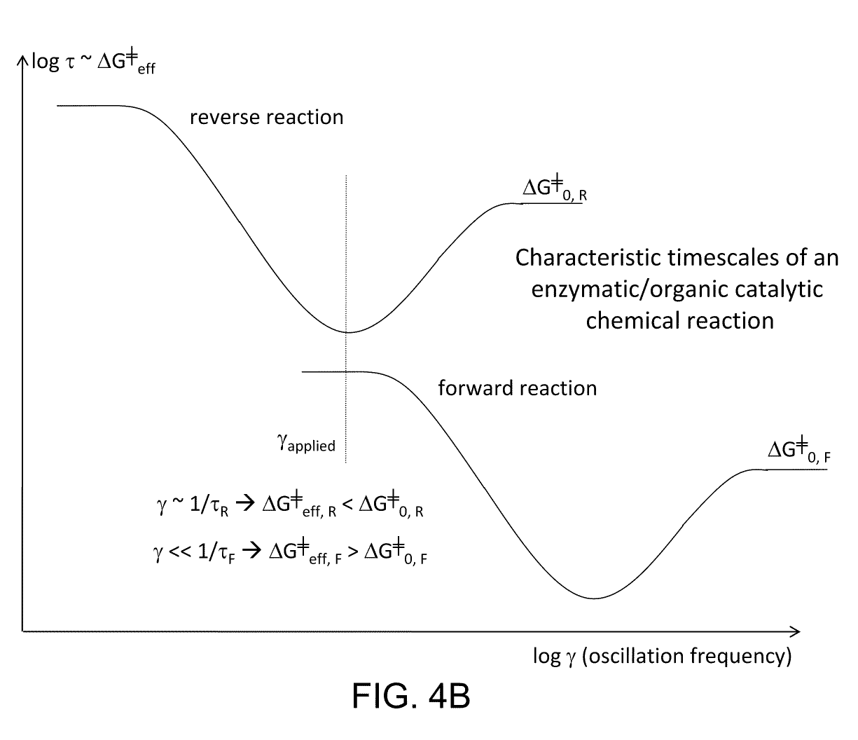

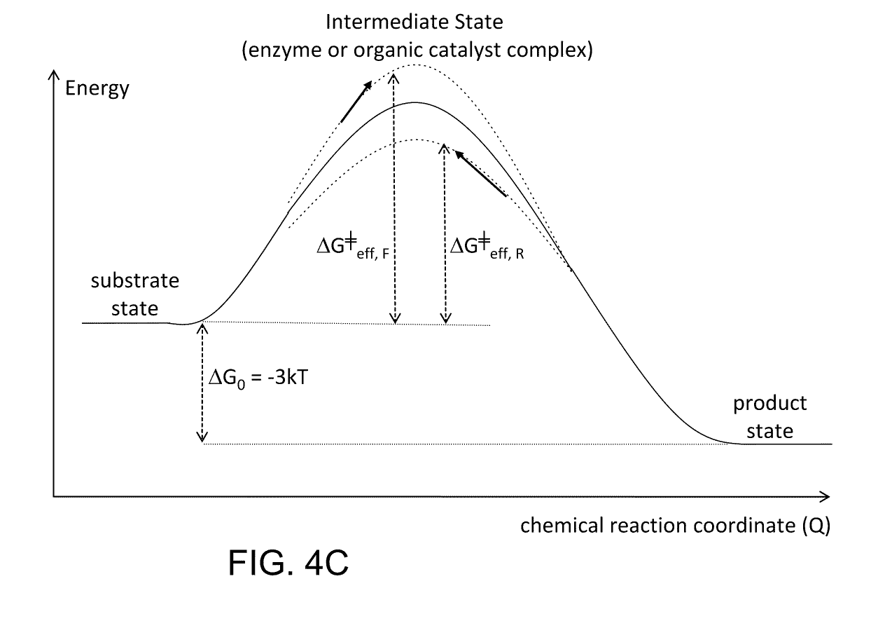

Finally, if γ˜(1/τ), that is, if the oscillation is of the same order of magnitude as the characteristic timescale of the transition, then there will be a resonant effect between the impacts of the applied field (i.e., the driving force) and the restoring transitions (also called “resonant activation”), which will increase the transition probability, and therefore, decrease the effective barrier height. The effective barrier heights for the three regimes are qualitatively summarized in FIG. 4A.

As shown in FIG. 4B, for the example discussed earlier, the frequency range of resonant activation for the forward reaction corresponds to the forward characteristic timescale (γ˜1/τ_{F}˜exp(−5)). Similarly, the range of resonant activation for the reverse reaction occurs at a lower frequency, i.e., a higher barrier, and thus, a higher characteristic timescale (γ˜1/τ_{R}˜exp(−8)).

Therefore, as the plots for the three regimes of frequency dependence for the forward and reverse reactions illustrate, by controlling the oscillation frequency of the electric field, the effective barrier heights for the forward and reverse reactions can be affected differently. For example, for an oscillation frequency that overlaps the resonant activation range of the reverse reaction (γ˜1/τ_{R}), the effective barrier height for the reverse and forward reactions will decrease and increase, respectively, compared to their non-perturbed states with no field.

As shown in FIG. 4C, the result is a hysteresis loop for an enzymatic/organic catalytic chemical reaction in which the forward and reverse reactions are affected differently (or by different amounts). The final equilibrium concentrations of the substrates and products, [S]_{final} and [P]_{final}, will change even though free Gibbs energy (ΔG) of the overall reaction was untouched. In this scenario, the applied field will do work against the final equilibrium concentrations, i.e., [S]_{final}/[P]_{final}≠[S]_{eq}/[P]_{eq}. Thus, directionality of a chemical reaction (e.g., chemical hysteresis) can be altered using a non-directional force.

The static and kinetic effects (i.e., the frequency independent and frequency dependent aspects of the ASYS mechanism), can be used, either together or separately, to manipulate enzymatic or organic catalytic chemical reactions, achieve catalysis, manipulate final concentrations of substrates and products, or manipulate certain paths over others in a chemical reaction with multiple possible paths to proceed, among other things. In other words, we can use a non-directional force (in time or in chemical potential) to do directional work (in the sense of chemical reaction energetics).

As an example implementation, consider an enzyme-catalyzed reaction, where the intermediate state is preferentially stabilized by the electrostatic interactions between amino residues of the enzyme and the substrate, and therefore, must be sensitive to sufficiently strong external electric fields (for a review: Villa and Warshel, “Energetics and dynamics of enzymatic reactions”, Journal of Physical Chemistry B, vol 105, pp 7887-7907, 2001). Further assume the enzyme is covalently attached to a surface (e.g. FIG. 6C) and is exposed to a buffer solution with total ion concentration in the range of 1 to 100 mM. When such a surface is charged by an external voltage source, a diffuse electrical double layer is formed (e.g., Israelachvili, “Intermolecular and surface forces”, Academic Press, San Diego, 2000). The thickness of the electrical double layer is very sensitive to the ion concentration in the buffer and the charge density on the surface (the charge density is determined by the magnitude of applied potential); typical thickness of the electrical double layer varies from 1 Å to ˜100 nm depending on the above two parameters. Such a small thickness means that the applied potential falls off over very short distances, generating very high electric fields within the double layer.

FIG. 5A shows the theoretical dependence of the electric field as a function of the distance from the surface for three different ion concentrations, suggesting electric field magnitudes on the order of 10⁷ V/m can be achieved within a few nanometers from the surface using relatively low potential values (<1 V).

FIG. 5B shows a simple setup, where an enzyme 30 attached to the surface 32 is exposed to strong electric fields. Note that the change of the sign of the applied potential will lead to the reversal of the electric field direction. Thus by varying the applied potential 34, the enzyme can be exposed to a time-dependent electric field with virtually any time profile, modulated at a variable frequency.

Variations of the attached enzyme 100 for this application can be viewed in FIG. 6C. The enzyme, 100, is attached to the surface, 104. The modulation 106 creates the perturbation on the enzyme, 100, and substrate, 102, pair. The result of the perturbation can be enhanced using modifications to the enzyme such as attached metal beads, 108, or attached polymers, 110, that may be more responsive to the perturbation.

In many cases, the apparatus can be implemented in a wide variety of kinds of computing hardware, software, firmware, or combinations of them, in many cases with the aid of a wide variety of communication networks, user interfaces, interface devices, operating systems, databases, processes, process control and monitoring systems, and user applications. FIG. 1 illustrates an example apparatus 40 with respect to an enzymatic chemical reaction.

FIG. 6A shows an example apparatus 73 to implement the system described above. An enzymatic chemical reaction chamber 74 is operated as a capacitor, with two electrodes 75 on its surface. Chemical reactants 76 are injected from the left into the reaction chamber and extracted from the chamber from the right as products out 77. An electrical source 78 provides a sinusoidal voltage to be applied to the reaction chamber to create a sinusoidal electric field across the capacitor plates. The chamber is coupled in parallel to an electrical inductor 79. The resonant frequency of the capacitor-inductor pair is matched to the frequency of the input electric field. Because of this matching, a significant amount of the applied electrical energy can be recycled in the LC-circuit, and the energy efficiency of the system can be significantly improved.

FIG. 6B shows another example apparatus 91 to implement the system described above. As in FIG. 6A, the enzymatic chemical reaction chamber 93 is treated as a capacitor, with electrodes 95 on its surface to apply an electric field across the capacitor plates. The chemical reactants 97 are injected from the left into the reaction chamber and extracted from the right 99. An electrical source 101 provides the voltage applied across the reaction chamber, and the chamber is coupled in parallel to an electrical inductor 103, with the resonant frequency of the LC-circuit matched to the input frequency such that a significant amount of the electrical energy is circulated and the energy efficiency is improved.

Unlike the example of FIG. 6A, however, an additional second input is provided in the form of a mechanical force 105. If we assume an enzyme at a transition state is attached to the surface of the reaction chamber, a mechanical force (e.g. a pressure wave, via an acoustic transducer), for example, can be applied to the surface of the reaction chamber. In this example, the frequency of this second input is the same as the frequency of the electric field; however, the electric field lags the mechanical force by a 90-degree (i.e. π/2) phase delay, such that a counter-clockwise modulation is achieved in the energy-Q space.

In some examples, let's assume we have a chemical system (which we define very broadly and includes, for example, but is not limited to, any enzymatic or organic catalytic chemical reaction having one type of molecule in a left energy well 50 (molecule type A), another type of molecule in a right energy well 52 (molecule type B), and an energy barrier 54 between the two wells) (FIG. 7A).

Also assume that we apply a sinusoidal electric field 55 that modulates a potential energy of a transition state 56 of the molecules in the enzymatic or organic catalytic chemical reaction (FIG. 7B). Although the field has an effect on the transition state, the effect of the modulated electric field on the potential energies of the energy minima of the left well and of the right well approaches zero. At any point along the Q-axis, if we take the average of the force applied by the electric field to the system over time, we get zero, which means the electric field input is non-directional in time. Note that in this application, we often refer to any input that modulates the transition state vertically (e.g., the energy level of the transition state) as an electric field, even though strictly speaking, the input that modulates the energy level of the transition state need not be electrical; it could be a wide variety of other inputs. For example, given a particular system, it may not even be possible for an electric field to modulate the energy level of the transition state.

Now, let's assume the chemical system has the following asymmetry: the energy level 62 of the left well is higher than the energy level 64 of the right well (FIG. 7C), i.e. the free energy, H₀, of the chemical reaction is not zero.

FIG. 7D shows an example of how an input sinusoidal electric field changes a potential energy surface 66 of a chemical system that has an asymmetry, for example, the one shown in FIG. 7C.

In such an enzymatic or organic catalytic chemical reaction, the population of molecules at a given energy level is governed by a Boltzmann distribution. The energy levels of the molecules at various locations along the Q-axis (and thus, the populations of molecules at those locations along the Q-axis) are subject to thermal fluctuations, which constitute a perturbation to the system. In other words, the system is perturbed by thermal fluctuations.

FIG. 7E shows that, because of the asymmetry (e.g., in this example, H₀ is not equal to zero), the densities of states, the energy levels, and thus, the population distribution profiles of the molecules in the two wells change by different amounts in response to the modulation of the applied sinusoidal electric field. When the population distribution of one of the wells changes (e.g., is driven into non-equilibrium), the time it takes for the distribution to reach equilibrium again (to satisfy Boltzmann statistics) is dependent on the magnitude of the change. As such, at a given frequency of the applied sinusoidal electric field, the two wells may exhibit different responses to the input signal (e.g., different time constants to restore the population distributions in the respective wells back to equilibrium), which may lead to a nonzero relative phase lag (to restore equilibrium in the population distributions) between the two wells. Such a phase lag may have different impacts on the effective barrier heights, on the path lengths 70 and 72 of the forward and reverse paths along the Q dimension, and/or on the average populations of molecules in the two wells.

In another example, a system does not have an intrinsic asymmetry in free energy of the enzymatic or organic catalytic chemical reaction (H₀) or in Q space, i.e., H₀ and Q₀ are equal to zero (FIG. 9A). And there are two inputs: a square wave electric field 80 modulating a potential energy of the transition state, and a square wave mechanical force 82 modulating a location of the transition state (FIG. 9C). In this example, we assume the two inputs are at the same frequency and that transitions of the electric field 84 lag transitions of the mechanical force 86 by a 90-degree (i.e. π/2) phase delay (FIG. 9B).

FIG. 9C shows how the two inputs modulate the potential energy surface. The modulation proceeds in a counter-clockwise loop 88 in the energy & Q-space, and the existence of a direction of the loop comprises an asymmetry in the system, e.g., the asymmetry is externally applied and the system is now time-variant. A wide variety of other pairs of modulation could be used to provide a loop having a direction. If the mechanical force lagged the electric field, for example, then a clockwise loop would result. And if the modulations of the two inputs were sinusoidal (rather than square) waves, then the loop would be elliptical (rather than rectangular).

FIG. 9D shows the results of a simulation, illustrating the normalized population distribution (on the vertical axis), as a function of the chemical reaction coordinate (Q-axis), at a common frequency of the two inputs, in this case for a counter-clockwise, rectangular loop (e.g., the electric field lags the mechanical force and both are square waves). As in some of the previous examples, the population distribution along the Q-axis, and thus, the time-averaged yield of a chemical reaction, can be changed with such a modulation.

A wide variety of other examples, implementations, and applications can make use of the same or similar principles.

A system with an enzymatic chemical reaction and/or an organic catalytic chemical reaction may be perturbed by an influence (when we use the word force in our discussion, we mean to include any kind of influence such as a force or a perturbation) that is internal or external or a combination of the two. This perturbation in non-directional with respect to a variable of the system such that the average of the force applied by the perturbation is zero. As a result of the perturbation and an asymmetry of the perturbed system, a directional effect is caused with respect to the enzymatic or organic catalytic chemical reaction.

The force may be varying in space (spatial), in time (temporal), or both (spatio-temporal).

The force may be deterministic or stochastic. If deterministic, it may have a profile that is sinusoidal, square wave, piecewise linear or a different profile, including an arbitrary profile. It may also have a profile that is a weighted sum of the profiles listed previously. And if stochastic, it may have a Gaussian, Poisson, Lorentzian or a different statistical profile.

The force profile may be symmetric (f(x+n)=f(−x+n)), antisymmetric (−f(x+n)=f(−x+n)) or asymmetric.

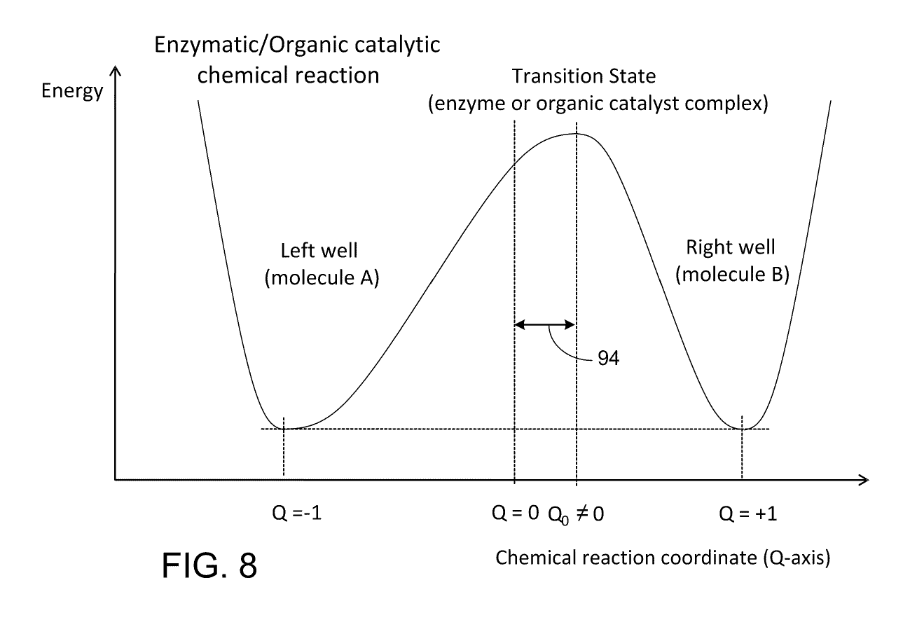

The force and/or the system's response to the force may be centered at zero or it can be biased 94 to a non-zero point (FIG. 8). A second force may be applied or various environmental parameters or other influences (e.g., temperature, pressure, volume) may be changed to shift or bias the system response.

The force may be applied as a digital or an analog signal.

The applied force profile may vary with only one parameter (FIG. 3B), with two parameters (FIG. 9B), or with more than two parameters. How a two-parameter loop can be used to increase the maximum theoretical efficiency of a Brownian motor or pump is described in [Parrondo, Blanco, Cao and Brito, “Efficiency of Brownian motors”, Europhysics Letters, vol 43, no 3, pp 248-254, 1998].

The applied force may be in the form of an electric field, a magnetic field, substrate concentration, product concentration, pH, pressure, temperature, acoustic field, electromagnetic field, or any other force type to interact with any part(s) of a chemical system and modulate its energy levels.

The applied force may be oscillating at a frequency. The frequency of oscillation may be controlled to cause, bias and/or manipulate the directional effect. Perturbing the system by altering an energy barrier to the enzymatic/organic catalytic chemical reaction includes shifting the energy barrier along the energy axis and/or along the Q-axis (chemical reaction coordinate). The system is characterized by an asymmetry such that the system response is directional in response to a nondirectional perturbation. The effect that the asymmetry has on the system response is related to the frequency of the perturbations and the forward and reverse rates of the enzymatic chemical reactions. The directional effect is an effect on a reaction rate and/or yield of the enzymatic chemical reaction. The directional effect is used to achieve at least one of the following: manipulating enzymatic/organic catalytic chemical reactions, achieving catalysis, manipulating final concentrations of substrates and/or products, and manipulating paths of the enzymatic/organic catalytic chemical reaction.

The asymmetry for the ASYS mechanism may be intrinsic to the system or applied extrinsically to the system or a combination of the two.

The asymmetry may have a spatial, a temporal, a spatio-temporal, or an energetic nature.

The asymmetry may be permanent or it may be induced or a combination of the two.

The enzymatic/organic catalytic chemical reaction may be a surface reaction, a bulk reaction, or a membrane reaction, or combinations of them.

The enzymatic chemical reaction may be an organic catalytic chemical reaction.

The enzymatic/organic catalytic chemical reaction may be a spontaneous reaction (exothermic) or a non-spontaneous reaction (endothermic), or a combination of the two.

The enzymatic/organic catalytic chemical reaction may comprise a single chemical path or it may have multiple possible paths to proceed.

The enzymatic/organic catalytic chemical reaction may be manipulated by controlling the direction of the reaction, by changing a substrate concentration and/or a product concentration or a ratio of concentrations, by doing work on the system that the system would otherwise not do in the absence of the motor or pump (e.g., against or along other forces and/or gradient), by catalyzing the reaction, or by specific enhancement and/or suppression of reactions and/or paths.

The enzymatic/organic catalytic chemical reaction may be a single step reaction or a multi-step reaction. It may have a single pathway or multiple paths. These multiple paths may be proceeding sequentially or in parallel.

The applied force may be enhanced, e.g., by running the enzymatic/organic catalytic chemical reaction close to or on a surface, and thereby, enhancing the electric field strength and/or the effective pH (its absolute value and/or its gradient) within the electric double layer that forms on the surface.

When the motor or pump is in the form of a Q-asys, the Q-asys may be optimized for work done (e.g., by optimizing the frequency of oscillation and the field magnitude to maximize the change in effective barrier height and/or to maximize the change in final equilibrium concentration of a particular molecule), for energy efficiency (e.g., by applying a two-or-more-parameter loop profile for the external force to reduce the irreversibility of the transitions), or for operation in a desired regime (e.g., by applying a second force to bias/shift the system response, or for a particular load amount). When applying two or more parameters with a loop profile, there is a particular phase relationship between the parameters (FIGS. 9A, 9B, 9C), and the asymmetry of interest is the direction of the loop (i.e. clockwise or counterclockwise).

The interaction of the force with the system may be enhanced by employing influence mediators. These influence mediators may be genetically targeted (e.g., mutated enzymes), artificially attached (e.g., by attaching beads or certain chemical groups to enzymes), or naturally occurring modifications (e.g., post-translational modifications of proteins). These influence mediators may be used together with the Q-asys method, or they may be used alone.

The Q-asys may be applied towards manipulations of enzymatic or organic catalytic chemical reactions in the following ways, among others:

The reaction may be manipulated, so that the final substrate concentration, final product concentration, and/or the ratio of the two concentrations may be altered to be different from their equilibrium concentrations, i.e. [S]_{final}≠[S]_{eq}, [P]_{final}≠[P]_{eq}, or [S]_{final}/[P]_{final}≠[S]_{eq}/[P]_{eq}, respectively.

The spontaneity of the reaction may be increased, decreased, or reversed, i.e., ΔG may be decreased, increased or its sign changed, respectively.

The yield of a specific path and/or product may be manipulated, where the probability of a specific pathway and/or product, relative to another alternative pathway or product, may be increased or decreased.

The Q-asys may comprise multiple chemical steps, or multiple chemical pathways. There may be multiple forces involved.

The Q-asys may be applied towards catalysis of enzymatic and organic catalytic chemical reactions in the following ways, among others:

A catalyst may be enhanced further. The catalyst present in the reaction may be a surface catalyst or a bulk catalyst; it may be an enzyme (membrane or bulk) or an organic catalyst.

A catalytic reaction may be enhanced further (and not necessarily by enhancing the catalyst present in the reaction).

An enzymatic or organic catalytic reaction may be catalyzed without a catalyst molecule or surface present; that is, a catalyst may be emulated.

An enzymatic or organic catalytic chemical reaction, step of a reaction, a chemical path and/or a catalyst may be speeded up or slowed down. They each may be turned on or turned off.

The specific mechanism of catalysis may be via the manipulation of an energy barrier, manipulation of a transmission coefficient, modulation of an affinity, or manipulation of the concentrations of a substrate, product, and/or an intermediate state.

New mixtures and/or products may be obtained from using such Q-asys in catalysis, catalytic reactions and/or towards manipulation of catalysts. As such, these mixtures and/or products are also claimed in this application.

A result or an outcome of Q-asys may be used in various industries and application, including, but not limited to, chemical manufacturing and/or processing, industrial, energy, biological, or other fields of chemistry, biology, and/or biochemistry.

In general, the apparatus includes a site for an enzymatic/organic catalytic chemical reaction, and a device controlled to perturb a system that includes the enzymatic/organic catalytic chemical reaction, the average of the force applied by the perturbation over time being zero and a directional effect being caused with respect to the chemical reaction as a result of the perturbation and an asymmetry of the perturbed system. Implementations may include one or more of the following features. The device can include a controlled voltage, current, temperature, pressure, pH or concentration perturbations. The site can be a surface with an enzyme covalently attached it. The controlled voltage causes a diffuse electrical double layer to be formed at the site. The controlled voltage induces an electric field magnitude in the double layer on the order of 10⁷ V/m. The controlled voltage can induce a time-dependent electric field having any arbitrary time profile and frequency.

Other implementations and applications are also within the scope of the following claims, and other claims.

1. A method comprising
applying a perturbation to a system comprising an enzymatic chemical reaction and/or an organic catalytic chemical reaction, the perturbation being non-directional (average of the force applied by the perturbation being zero) with respect to a variable of the system, and
causing a directional effect with respect to the said enzymatic or organic catalytic chemical reaction as a result of the perturbation and an asymmetry of the perturbed system.

2. The method of claim 1 in which the perturbation is internal to the system or applied externally to the system.

3. The method of claim 1 in which the perturbation is varying in space or in time or in time and space both.

4. The method of claim 1 in which the perturbation is deterministic or stochastic.

5. The method of claim 4 in which the perturbation has a profile that is sinusoidal, square wave, piecewise linear, or arbitrary, or a weighted sum of a combination of sinusoidal, square wave and piecewise linear or arbitrary.

6. The method of claim 4 in which the perturbation distribution function has a statistical profile that is Gaussian, Poisson, Lorentzian, or continuous probability distribution or discrete probability distribution.

7. The method of claim 1 in which the perturbation has a profile that is symmetric or antisymmetric or asymmetric.

8. The method of claim 1 in which the perturbation and/or the system response to the perturbation is centered at zero or biased to a non-zero value.

9. The method of claim 8 in which an influence or an environmental parameter biases the perturbation and/or the system response.

10. The method of claim 1 in which the perturbation is applied as a digital signal or an analog signal.

11. The method of claim 1 in which the perturbation is applied as a profile that may vary with one parameter, with two parameters, or with more than two parameters.

12. The method of claim 1 in which the perturbation may be in the form of at least one of: an electric field, a magnetic field, a substrate concentration, a product concentration, pH, pressure, temperature, an acoustic field, or an electromagnetic field, in order to interact with a part of a chemical system and to modulate its energy levels.

13. The method of claim 1 in which the asymmetry is intrinsic to the system, extrinsic to the system, or both intrinsic and extrinsic to the system.

14. The method of claim 1 in which the asymmetry comprises spatial, temporal, spatio-temporal, or energetic.

15. The method of claim 1 in which the asymmetry comprises permanent or induced or a combination of the two.

16. The method of claim 1 in which the said chemical reaction comprises a surface reaction, a bulk reaction, or a membrane reaction, or a combination of two or more of these.

17. The method of claim 1 in which the said chemical reaction comprises a spontaneous reaction (exothermic) or a non-spontaneous reaction (endothermic), or a combination of these two.

18. The method of claim 1 in which the said chemical reaction comprises a single chemical pathway or multiple chemical pathways.

19. The method of claim 1 in which the said chemical reaction is manipulated by controlling a direction of the reaction.

20. The method of claim 1 in which the said chemical reaction is manipulated by changing a substrate concentration or changing a product concentration or changing a ratio of concentrations.

21. The method of claim 1 in which the said chemical reaction is manipulated by doing work on the system that the system would otherwise not do, including against or along other influences and/or gradients.

22. The method of claim 1 in which the said chemical reaction is manipulated by catalyzing the reaction.

23. The method of claim 1 in which the said chemical reaction is manipulated by applying one or more of specific enhancement of pathways, specific enhancement of reactions, specific suppression of pathways and specific suppression of reactions.

24. The method of claim 1 in which the said chemical reaction comprises a single step reaction or a single pathway or a multiple step reaction or multiple pathways.

25. The method of claim 24 in which the multiple pathways are sequential or parallel.

26. The method of claim 1 in which the perturbation is enhanced by running the said chemical reaction on a surface or close to a surface.

27. The method of claim 1 in which the perturbation is optimized for magnitude of the work done, for energy efficiency, and/or for a particular load amount.

28. The method of claim 27 in which the optimization for work done comprises optimizing a frequency of oscillation and/or a field magnitude of the perturbation to maximize a change in effective barrier height.

29. The method of claim 27 in which the energy efficiency is optimized by applying a loop profile comprising two or more parameters for the influence.

30. The method of claim 29 in which there is a particular phase relationship between the parameters.

31. The method of claim 1 in which the perturbation is optimized for operation in a desired regime.

32. The method of claim 31 in which the operation in a desired regime is optimized by applying a second perturbation to bias or shift the system response.

33. The method of claim 1 in which application of the perturbation is enhanced by influence mediators.

34. The method of claim 1 in which the directional effect comprises altering relative to equilibrium concentrations a final substrate concentration, a final product concentration, or a ratio of a final substrate concentration and a final product concentration.

35. The method of claim 1 in which the directional effect comprises increasing, decreasing, or reversing spontaneity of the reaction.

36. The method of claim 1 in which the directional effect comprises changing a probability of a specific pathway and/or product, relative to an alternative pathway or product, to change a yield of the specific pathway and/or product.

37. The method of claim 1 in which the directional effect is applied to multiple chemical steps, or multiple chemical pathways.

38. The method of claim 1 in which the directional effect comprises accelerating or decelerating an enzymatic chemical reaction, an organic catalytic chemical reaction, a step of a reaction, a chemical pathway and/or a catalyst.

39. The method of claim 1 in which the directional effect of the perturbation comprises catalyzing an enzymatic chemical reaction or an organic catalytic chemical reaction.

40. The method of claim 39 in which the catalyzing comprises manipulating an energy barrier, modulating a transmission coefficient or an affinity, or manipulating a concentration of a substrate, product, and/or an intermediate state.

41. The method of claim 1 also including using a result or an outcome in chemical manufacturing, chemical processing, industrial application, energy application, biological application, field of chemistry, field of biology, and/or field of biochemistry.

42. An apparatus comprising
a site for an enzymatic or organic catalytic chemical reaction, and
a device controlled to perturb a system that includes the enzymatic or organic catalytic chemical reaction, the average of the force applied by the perturbation being zero, a directional effect being caused with respect to the chemical reaction as a result of the perturbation and an asymmetry of the perturbed system.

43. The apparatus of claim 42 in which the device comprises a controlled voltage, current, temperature, pressure, pH and/or concentration perturbations.

44. The apparatus of claim 42 in which the site comprises a surface.

45. The apparatus of claim 44 in which enzymes are covalently attached to the surface.
